(19) United States
(12) Patent Application Publication
(10) Pub. No.: US 2005/0272983 A1
(43) Pub. Date: Dec. 8, 2005

(54) Cellular handset resource utilization

(76) Inventor: Benjamin Maytal, Mevasseret Zion (IL)

Correspondence Address: DANIEL J SWIRSKY, PO BOX 2345, BEIT SHEMESH, 99544, IL

(21) Appl. No.: 11/145,173

(22) Filed: Jun. 6, 2005

Related U.S. Application Data
(60) Provisional application No. 60/577,493, filed on Jun. 7, 2004.

(51) Int. Cl. A61B 5/00

(52) U.S. Cl. 600/300

(57) ABSTRACT

The present invention will discuss how through the usage of the cellular handset or other host system short range communication, various portable devices and in particular medial monitoring devices can utilize the resources available inside the cellular handset or in other host system, and how communication can be established with a central service office. This will enable much smaller and cost effective portable devices. And the provision of quality service based on continuous monitoring of health conditions.

### CROSS-REFERENCE TO RELATED APPLICATIONS

This application claims benefit from U.S. Provisional Patent Application No. 60/577,493, filed Jun. 7, 2004, which is hereby incorporated by reference.

### FIELD OF THE INVENTION

The present invention relates to portable devices and in particular to portable medical monitoring devices, connected with host system, specifically cellular handset.

### BACKGROUND OF THE INVENTION

Portable medical monitoring devices are becoming more popular. New technologies allow low cost optical sensors, which allow non-invasive medical and fitness parameter monitoring. Wearable monitors are becoming feasible. They enable on going, 24 h monitoring of key health and fitness parameters. Each of the portable medical monitoring devices mentioned above typically incorporates a screen display and memory as well.

For best utilization, this information needs to be transmitted to a central service office. Cellular handsets are ubiquitous. There are more than 1 Billion handsets used worldwide. Cellular handset is by far the most popular device people are carrying with them everywhere.

A cellular handset (and other host systems, such as PC's and PDA's) incorporates in it certain capabilities: A screen display, memory, long range data communication (e.g. GPRS). Certain devices have cameras inside as well as GPS (Global positioning System)—worldwide coordinate finder. In many cases the cellular handset includes a speaker. Short range communication (IR, Bluetooth, wireless LAN) is becoming a standard. Short range communication is also available in PC's and PDA's.

Portable medical monitoring devices are in existence for several years (e.g. blood pressure monitors). New technologies, such as optical tomography, enable non invasive, on going monitoring of key health and fitness parameters. Some of the new portable devices, such as the Polar company heart rate watch, connect to cellular a cellular handset.

To the best of our knowledge, there are no portable devices with connection to a service provider. The exiting devices are autonomous and do not utilize the available resources of the host system.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 describes a cellular handset or any other host system 2 (e.g. PC or PDA) connected via short range communication media to a portable medical monitoring device 7 and through long range communication (e.g. GPRS, ADSL) to a central server office 8. It includes long range voice and data communication block 1 connecting to service, a CPU 2, Memory 4, Screen display 4 and short range (e.g. Bluetooth) communication block 3. It can potentially include GPS (Global Positioning System) 6 as well. The portable medical monitoring device may be a wearable device.

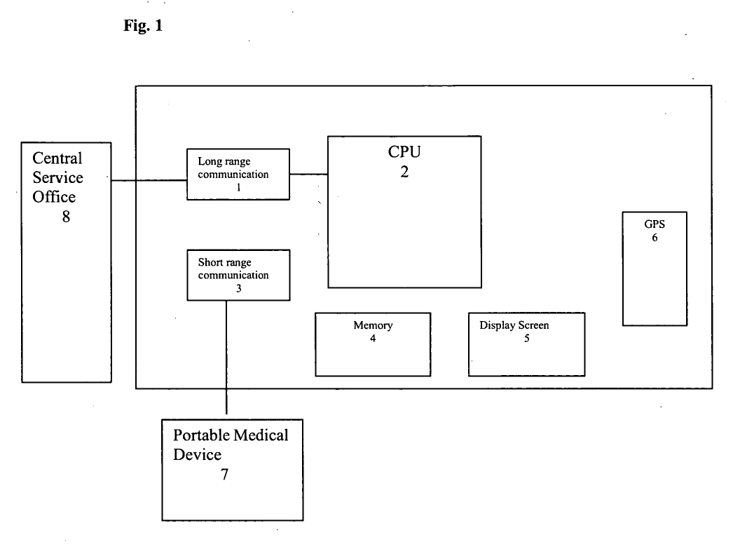

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements

### SUMMARY OF THE PRESENT INVENTION

The present invention will describe how a host system can be connected to the portable device, specifically a medical monitoring device and also to a service provider, and pass relevant information from the monitoring device to the service provider and vice versa. It will also describe how the portable device can be made more cost effective and flexible by using resources of the host system, specifically a cellular handset.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The host system will get relevant data from the portable device over wireless short range communication and will transmit it via the long range communication media to the central service office.

A special portable medical monitoring device control software can be loaded in the cellular handset CPU or any other host system (e.g. PC or PDA). As part of the user menu, the end user will be able to activate the portable device. (e.g. perform a blood pressure test).

The software can be loaded prior to the handset shipment or can be loaded to it once it is in the field (e.g. Java software).

In order to save power, the portable device will be in standby mode and will wake up upon detection on incoming signal over the short range communication channel.

The handset can read relevant information from the portable device and display it on screen. For example, the heart rate or the temperature. It can store information in its internal memory. It can transmit information from the portable device over the long range data communication to a central service office 8 or over the web (the internet) to interested parties/care takers. The information transmission to the central service office can be done either periodically or by request from the center or upon detection of a predetermined emergency case. It can use the internal speaker to send alarm sounds in case the user is in trouble (e.g. heart rate dropped below a certain range). It can also generate SMS messages and email messages. It can download information (e.g. music) and load it into the portable device as well.

In a different mode of operation, the cellular handset may be woken up by the portable device. For example, for medial devices monitoring the health of an end user, the medical monitoring device may detect an emergency case and transmit the information to the cellular handset. It will send a signal over the short range communication channel which will wake up the handset or will wake up the short range communication for the handset. The handset will receive the data and then transmit it over the long range data communication. The cellular handset can send together with the information the positioning data derived form it's GPS (Global Positioning System) device.

Another problem portable devices are having is the power source. Batteries are expensive and heavy. Cellular handsets and PDA's are easily charged.

A new technology, called RFID has been developed in recent years. It allows transmission of power of radio frequency waves A cellular handset can be built with RFID transmission capabilities. A portable device can either rely entirely on RFID energy or partially.

Alternatively, a special connector can be applied to connect between the cellular handset and the portable device to charge the battery of this device.

The medical sensor may not send the cellular handset calculated parameters. It may just transfer raw samples. For example, from optical sensors. The actual calculation of the parameters will be done at the host—this will significantly reduce the CPU power required in the medical monitor and will allow adding more calculated parameters which are based on raw samples. As an example, calorie burn and intake can be calculated based on the heart rate. Alternatively, the host system may send the information to the central office, where the calculation will be done.

Please not that the features described above can be obtained not just in a cellular handset, but in any host system the medical

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A system comprising of a host system and portable medical measurement device connected to the host system through wireless short range communication and connected through a long range communication to a service center.

2. A system according to claim 1 where the host system is a cellular handset.

3. A system according to claim 1 where the information will be sent to the central office periodically.

4. A system as in claim 1 where the data will be sent to the central office upon request of the central office.

5. A system according to claim 1 where the portable medical monitoring device will be activated by the wireless short range communication initiation by the host system.

6. A system according to claim 1 where the host system will be activated by the wireless short range communication initiated by the portable medical monitoring device.

7. A system according to claim 1 where the portable device will transmit raw samples to the host system and the host system will calculate the parameters.

8. A system according to claim 1 where the raw samples will be passed to the central service office where the parameter calculation will be done.

9. A system according to claim 1 where the screen of the host system will be used for display of results.

10. A system according to claim 1 where the host system memory will be used to store results.

11. A system according to claim 2 where the cellular handset will also comprise a positioning system and will transmit the location information together with the medical information

12. A system according to claim 1 where specific measurements can be activated through a user menu.

13. A system according to claim 1 where emergency messages will be sent to a central office upon detection of predefined conditions.

14. A system according to claim 1 where an alarm will be sound upon detection of predefined conditions.

15. A system according to claim 1 where power will be transmitted to the portable device via the short range wireless communication media.

16. A system according to claim 1 where the portable device power source can be charged via a connector by the host system.

17. A system according to claim 1 where the portable device can transmit information over SMS or emails.

18. A system according to claim 1 where information from the central service office can be downloaded to the portable medical monitoring device.
